SEQUENCE LISTING (20 sequences)

SEQ ID NO: 1 - skipped

SEQ ID NO: 2 - protein, 5 aa, synthetic construct
  REGION 1..5: note = P block
  SITE 4: note = MISC_FEATURE - X may be any amino acid
VPGXG

SEQ ID NO: 3 - protein, 5 aa, synthetic construct
  REGION 1..5: note = G block
VGGVG

SEQ ID NO: 4 - protein, 5 aa, synthetic construct
  REGION 1..5: note = P block
  SITE 4: note = MISC_FEATURE - X is Val or Phe
VPGXG

SEQ ID NO: 5 - protein, 196 aa, synthetic construct
  REGION 1..196: note = GPG1
MKLVGGVGVGGVGVGGVGVGGVGVGGVGLWLGSGVPGVGVPGVGVPGFGVPGVGVPGVGVPGVGVPGVGVPGFGVPGVGVPGVGVPGVGVPGVGVPGFGVPGVGVPGVGVPGVGVPGVGVPGFGVPGVGVPGVGVPGVGVPGVGVPGFGVPGVGVPGVGKLVGGVGVGGVGVGGVGVGGVGVGGVGLWLEHHHHHH

SEQ ID NO: 6 - protein, 205 aa, synthetic construct
  REGION 1..205: note = GPG3
MKLVGGVGVGGVGVGGVGVGGVGVGGVGLWLGSGVPGVGVPGVGVPGFGVPGVGVPGVGVPGVGVPGVGVPGFGVPGVGVPGVGVPGVGVPGVGVPGFGVPGVGVPGVGVPGVGVPGVGVPGFGVPGVGVPGVGVPGVGVPGVGVPGFGVPGVGVPGVGKLVGGVGVGGVGVGGVGVGGVGVGGVGLWLEHHHHHHKAAKGRGDS

SEQ ID NO: 7 - protein, 6 aa, synthetic construct
  REGION 1..6: note = Linker 1
LWLGSG

SEQ ID NO: 8 - protein, 4 aa, synthetic construct
  REGION 1..4: note = RGDS
RGDS

SEQ ID NO: 9 - protein, 8 aa, synthetic construct
  REGION 1..8: note = LPGFPGLK
LPGFPGLK

SEQ ID NO: 10 - protein, 8 aa, synthetic construct
  REGION 1..8: note = LPGFPGTP
LPGFPGTP

SEQ ID NO: 11 - protein, 9 aa, synthetic construct
  REGION 1..9: note = GPPGLSGPP
GPPGLSGPP

SEQ ID NO: 12 - protein, 8 aa, synthetic construct
  REGION 1..8: note = FPGPPGPP
FPGPPGPP

SEQ ID NO: 13 - protein, 8 aa, synthetic construct
  REGION 1..8: note = LPGLPGPP
LPGLPGPP

SEQ ID NO: 14 - protein, 8 aa, synthetic construct
  REGION 1..8: note = FPGLPGPP
FPGLPGPP

SEQ ID NO: 15 - protein, 10 aa, synthetic construct
  REGION 1..10: note = GPPGPPGSPG
GPPGPPGSPG

SEQ ID NO: 16 - protein, 8 aa, synthetic construct
  REGION 1..8: note = LPGPPGPP
LPGPPGPP

SEQ ID NO: 17 - protein, 9 aa, synthetic construct
  REGION 1..9: note = FPGSPGFPG
FPGSPGFPG

SEQ ID NO: 18 - protein, 9 aa, synthetic construct
  REGION 1..9: note = GSPGLPGTP
GSPGLPGTP

SEQ ID NO: 19 - protein, 8 aa, synthetic construct
  REGION 1..8: note = IGLSGEKG
IGLSGEKG

SEQ ID NO: 20 - protein, 4 aa, synthetic construct
  REGION 1..4: note = REDV
REDV

(19) United States
(12) Patent Application Publication
(10) Pub. No.: US 2025/0243453 A1
(43) Pub. Date: Jul. 31, 2025

(54) CELL CULTIVATION OR PROLIFERATION SUBSTRATE

(71) Applicant: NATIONAL UNIVERSITY CORPORATION TOKAI NATIONAL HIGHER EDUCATION AND RESEARCH SYSTEM, Aichi (JP)

(72) Inventors: Ayae NARUTAKI, Aichi (JP); Chikara OHTSUKI, Aichi (JP); Jin NAKAMURA, Aichi (JP); Kazunori HAYASHI, Aichi (JP)

(73) Assignee: NATIONAL UNIVERSITY CORPORATION TOKAI NATIONAL HIGHER EDUCATION AND RESEARCH SYSTEM, Aichi (JP)

(21) Appl. No.: 18/854,312

(22) PCT Filed: Mar. 20, 2023
(86) PCT No.: PCT/JP2023/010893

§ 371 Date: Oct. 4, 2024

(30) Foreign Application Priority Data
Apr. 7, 2022 (JP) 2022-063856

(51) Int. Cl. C12N 5/00

(52) U.S. Cl. CPC C12N 5/0068; C12N 2513/00

(57) ABSTRACT

Provided is a cell culture or growth substrate that enables cells to be separated more easily without the use of a drug.

A cell culture or growth substrate comprising a composition in gel form or gel-forming sol form, or a dried product of the composition, the composition comprising a polypeptide comprising an elastin-like block peptide sequence that comprises a G sequence block consisting of (SEQ ID NO: 1: X¹GGX²G)ₙ, wherein each X¹ is the same or different and represents V or L, each X² is the same or different and represents V or L, and n represents an integer of 4 or more, and a P sequence block consisting of (SEQ ID NO: 2: VPGX³G)ₘ, wherein each XV is the same or different and represents any amino acid, and m represents an integer of 5 or more.

### TECHNICAL FIELD

The present invention relates to a cell culture or growth substrate and the like.

### BACKGROUND ART

Three-dimensional cell culture is a technique for culturing cells in an artificially created three-dimensional environment. Three-dimensional cell culture can be expected to allow for a three-dimensional cell arrangement and interactions, and can mimic an environment closer to that in the body compared with two-dimensional cell culture. Thus, three-dimensional cell culture is attracting particular attention as a method for reproducing the high functions of biological tissue in a wide range of fields, including regenerative medicine, tumor biology, and drug discovery research. In recent years, cell sheets produced by two-dimensional cell culture have been used for regenerative medicine.

In three-dimensional cell culture, gels of the extracellular matrix, such as collagen, are used as cell culture or growth substrates. However, enzymatic digestion or the like is required to separate cells from a substrate after culture. In two-dimensional cell culture as well, enzymatic digestion or the like is required to separate cells from a substrate after culture.

Elastin is an important functional protein that imparts elasticity and stretchability to biological tissue; however, its use as a material is significantly delayed due to the difficulty of handling resulting from its high hydrophobicity. The present inventors have developed a double-hydrophobic elastin-mimetic polypeptide (GPG) that forms self-assembling nanofibers in water (Non-patent Literature (NPL) 1). The resulting fibers can form physical gels and can be expected to be applied to biomaterials.

### CITATION LIST

### Non-Patent Literature

NPL 1: D. H. T. Le, R. Hanamura, D.-H. Pham, M. Kato, D. A. Tirrell, T. Okubo, A. Sugawara-Narutaki, Self-Assembly of Elastin-Mimetic Double Hydrophobic Polypeptides, Biomacromolecules. 14 (2013) 1028-1034. https://doi.org/10.1021/bm301887m.

### SUMMARY OF INVENTION

### Technical Problem

An object of the present invention is to provide a cell culture or growth substrate that enables cells to be separated more easily without the use of a drug.

### Solution to Problem

The present inventors conducted extensive research to achieve the above object and found that the object can be achieved by a cell culture or growth substrate comprising a composition in gel form or gel-forming sol form, or a dried product of the composition, the composition comprising a polypeptide comprising an elastin-like block peptide sequence that comprises a G sequence block consisting of (SEQ ID NO: 1: X¹GGX²G)ₙ, wherein each X¹ is the same or different and represents V or L, each X² is the same or different and represents V or L, and n represents an integer of 4 or more, and a P sequence block consisting of (SEQ ID NO: 2: VPGX³G)ₘ, wherein each XV is the same or different and represents any amino acid, and m represents an integer of 5 or more. The inventors conducted further research on the basis of this finding and completed the present invention. Specifically, the present invention includes the following subject matter.

Item 1. A cell culture or growth substrate comprising a composition in gel form or gel-forming sol form, or a dried product of the composition, the composition comprising a polypeptide comprising an elastin-like block peptide sequence that comprises a G sequence block consisting of (SEQ ID NO: 1: X¹GGX²G)ₙ, wherein each X¹ is the same or different and represents V or L, each X²⁷ is the same or different and represents V or L, and n represents an integer of 4 or more, and a P sequence block consisting of (SEQ ID NO: 2: VPGX³G)ₙ, wherein each XV is the same or different and represents any amino acid, and m represents an integer of 5 or more.
Item 2. The cell culture or growth substrate according to Item 1, wherein the sequence structure of the elastin-like block peptide sequence is, from the N-terminal side, G sequence block-P sequence block-G sequence block, G sequence block-P sequence block-P sequence block-G sequence block, P sequence block-G sequence block, or G sequence block-P sequence block.
Item 3. The cell culture or growth substrate according to Item 1 or 2, for use in three-dimensional culture or three-dimensional growth of cells.
Item 4. The cell culture or growth substrate according to Item 1 or 2, for use in two-dimensional culture or two-dimensional growth of cells.
Item 5. The cell culture or growth substrate according to any one of Items 1 to 4, wherein the composition retains cells.
Item 6. The cell culture or growth substrate according to any one of Items 1 to 5, wherein the concentration of the polypeptide in the composition is 0.2 w/v % or more.
Item 7. The cell culture or growth substrate according to any one of Items 1 to 6, wherein the concentration of the polypeptide in the composition is 0.2 to 3 w/v %.
Item 8. The cell culture or growth substrate according to any one of Items 1 to 7, for use by solating the composition in gel form and/or for use by gelating the composition in sol form.
Item 9. A cell culture or growth method comprising culturing or growing cells using the cell culture or growth substrate according to any one of Items 1 to 8.
Item 10. A cell population obtainable by the cell culture or growth method according to Item 9.

### Advantageous Effects of Invention

The present invention provides a cell culture or growth substrate that enables cells to be separated more easily without the use of a drug.

### BRIEF DESCRIPTION OF DRAWINGS

FIG. 1 is a TEM image of the GPG1 gel obtained in Test Example 2. A photograph of a tube containing the gel is shown in the upper-right corner of the image.

FIG. 2 shows the rheological measurement results of Test Example 3. The vertical axis represents storage modulus (G′) and loss modulus (G″). The horizontal axis represents the elapsed time of the test (unit: s (seconds)).

FIG. 3 shows the rheological measurement results at the time of initial gelation in Test Example 4. A: time dispersion measurement (constant frequency of 1 Hz, constant strain of 1%), B: strain dispersion measurement (constant frequency of 1 Hz), and C: frequency dispersion measurement (constant strain of 1%).

FIG. 4 shows the rheological measurement results at the time of re-gelation (without cells) in Test Example 5. A: time dispersion measurement (constant frequency of 1 Hz, constant strain of 1%), B: strain dispersion measurement (constant frequency of 1 Hz), and C: frequency dispersion measurement (constant strain of 1%).

FIG. 5 shows the rheological measurement results at the time of re-gelation (with cells) in Test Example 6. A: time dispersion measurement (constant frequency of 1 Hz, constant strain of 1%), B: strain dispersion measurement (constant frequency of 1 Hz), and C: frequency dispersion measurement (constant strain of 1%).

FIG. 6 shows microscope images of Test Example 7. “In gel” shows an image of cells in a gel. “Recovery” shows an image of cells dispersed in PBS after solation.

FIG. 7 is a phase-contrast microscope image taken 7 days after the start of differentiation induction in Test Example 9.

FIG. 8 shows immunostaining images of a gel 7 days after the start of differentiation induction in Test Example 9. A shows a staining image of α-actinin, B shows a staining image of a cell nucleus, and C is a merged image of A and B.

### DESCRIPTION OF EMBODIMENTS

In the present specification, the terms “comprise,” “contain,” and “include” include the concepts of “comprising,” “containing,” “consisting essentially of,” and “consisting of.”

In the present specification, amino acid residues in an amino acid sequence may be represented, for example, by amino acids or specific amino acid names (e.g., valine and leucine). Amino acid residues in an amino acid sequence may also be represented by the one-letter code of amino acids.

In an embodiment, the present invention relates to a cell culture or growth substrate (which may be referred to as “the substrate of the present invention” in the present specification) comprising a composition in gel form or gel-forming sol form (which may be referred to as “the composition of the present invention” in the present specification) or a dried product of the composition, the composition comprising a polypeptide (which may be referred to as “the polypeptide of the present invention” in the present specification) comprising an elastin-like block peptide sequence that comprises a G sequence block consisting of (SEQ ID NO: 1: X¹GGX²G)ₙ, wherein each X¹ is the same or different and represents V or L, each X² is the same or different and represents V or L, and n represents an integer of 4 or more, and a P sequence block consisting of (SEQ ID NO: 2: VPGX³G)ₘ, wherein each X³ is the same or different and represents any amino acid, and m represents an integer of 5 or more. The following explains the cell culture or growth substrate.

The G sequence block is not particularly limited as long as it is a block consisting of a repeat sequence (X¹GGX²G)ₙ of the amino acid sequence (X¹GGX²G) set forth in SEQ ID NO: 1. The G sequence block is typically a block that can take a β-sheet structure. The G sequence block can impart the ability to form fibers by self-assembly to the polypeptide.

Each X¹ is the same or different and represents V or L, and each X² is the same or different and represents V or L. X¹ and X² are preferably V.

n represents an integer of 4 or more. When n is 4 or more, the polypeptide of the present invention can form a fibrous self-assembly. n is preferably 4 to 20, more preferably 4 to 12, even more preferably 4 to 8, and still even more preferably 4 to 6.

The P sequence block is not particularly limited as long as it is a block consisting of a repeat sequence (VPGX³G)ₘ of the amino acid sequence (VPGX³G) set forth in SEQ ID NO: 2. The P sequence block is typically a block that can take a β-turn structure. The P sequence block allows the polypeptide to have a lower critical solution temperature (LCST) and to self-assemble at the LCST or higher to phase separate from an aqueous solution.

The LCST of the polypeptide of the present invention (solvent: water, concentration: 0.03 wt %) is, for example, 10 to 30° C., and preferably 15 to 25° C.

Each X³ is the same or different and represents any amino acid. Examples of amino acids represented by X³ include amino acids having a basic side chain such as lysine, arginine, and histidine; amino acids having an acidic side chain such as aspartic acid and glutamic acid; amino acids having an uncharged polar side chain such as glycine, asparagine, glutamine, serine, threonine, tyrosine, and cysteine; amino acids having a nonpolar side chain such as alanine, valine, leucine, isoleucine, proline, phenylalanine, methionine, and tryptophan; amino acids having a β-branched side chain such as threonine, valine, and isoleucine; amino acids having an aromatic side chain such as tyrosine, phenylalanine, tryptophan, and histidine; and the like. The amino acids represented by X³ are preferably amino acids other than proline.

The LCST can be adjusted by the amino acids represented by X³. If the amino acids represented by X³ are hydrophilic amino acids, the LCST becomes too high, which is not preferable. From the viewpoint of easily adjusting the LCST to an appropriate (physiologically acceptable) temperature (e.g., 30 to 40° C.), it is preferred that some of the amino acids represented by X³ in the P sequence block are non-aromatic, hydrophobic amino acids, such as valine and leucine (preferably valine), and the other amino acids are aromatic, hydrophobic amino acids, such as phenylalanine and tryptophan (preferably phenylalanine). In this case, the proportion of the number of non-aromatic, hydrophobic amino acids represented by V relative to the total number of X³s in the P sequence block is, for example, 60 to 95%, preferably 70 to 80%, and more preferably 75 to 85%, and the proportion of the number of aromatic, hydrophobic amino acids represented by X³ relative to the total number of X³s in the P sequence block is, for example, 5 to 40%, preferably 20 to 30%, and more preferably 15 to 25%. Further, in this case, the aromatic, hydrophobic amino acids represented by X³ preferably appear as dispersed as possible in the P sequence block. For example, it is preferred that a repeat unit in which X³ represents an aromatic, hydrophobic amino acid appears at a ratio of one to, for example, one to seven repeat units (VPGX³G), preferably one to five repeat units (VPGX³G), more preferably two to four repeat units (VPGX³G), and even more preferably three repeating units (VPGX³G).

m represents an integer of 5 or more, m is preferably 10 to 100, more preferably 10 to 50, even more preferably 15 to 35, and particularly preferably 20 to 30.

The elastin-like block peptide sequence is not particularly limited as long as it comprises the G sequence block and the P sequence block. The number of G sequence blocks in the elastin-like block peptide sequence is not particularly limited, and is, for example, 1 to 5, preferably 2 to 5, more preferably 2 to 4, even more preferably 2 to 3, and particularly preferably 2. The number of P sequence blocks in the elastin-like block peptide sequence is not particularly limited, and is, for example, 1 to 5, preferably 1 to 4, more preferably 1 to 3, and even more preferably 1 to 2.

The blocks (the G sequence block and the P sequence block, the G sequence block and the G sequence block, and the P sequence block and the P sequence block) may be directly linked or may be linked via a linker sequence. The blocks are preferably linked via a linker sequence.

The linker sequence is not particularly limited as long as it does not significantly reduce the fiber-forming ability of the elastin-like block peptide sequence due to its self-assembly, and usually any amino acid or amino acid sequence can be used without significant limitation. The linker sequence is, for example, 1 to 20, preferably 1 to 15, more preferably 1 to 10, even more preferably 1 to 8, and still even more preferably 2 to 8 amino acids in length. Specific examples of the linker sequence include the amino acid sequence set forth in SEQ ID NO: 7: LWLGSG, the amino acid sequence set forth in KL, and the like. Furthermore, amino acid sequences in which one or more (for example, one to five, preferably one to three, more preferably one to two, and even more preferably one) amino acids are mutated (for example, substituted, deleted, inserted, and/or added, preferably substituted, more preferably conservatively substituted) relative to any of these amino acid sequences can also be used.

In the present specification, “conservative substitution” means the substitution of an amino acid with another amino acid having a similar side chain. For example, the substitution between amino acids having a basic side chain such as lysine, arginine, or histidine, is considered to be a conservative substitution. The following substitutions between other amino acids are also considered to be a conservative substitution: the substitution between amino acids having an acidic side chain such as aspartic acid or glutamic acid; the substitution between amino acids having an uncharged polar side chain such as glycine, asparagine, glutamine, serine, threonine, tyrosine, or cysteine; the substitution between amino acids having a nonpolar side chain such as alanine, valine, leucine, isoleucine, proline, phenylalanine, methionine, or tryptophan; the substitution between amino acids having a R-branched side chain such as threonine, valine, or isoleucine; and the substitution between amino acids having an aromatic side chain such as tyrosine, phenylalanine, tryptophan, or histidine.

From the viewpoint of the ability to form fibers by self-assembly, the sequence structure of the elastin-like block peptide sequence is preferably, from the N-terminal side, G sequence block-P sequence block-G sequence block, G sequence block-P sequence block-P sequence block-G sequence block, P sequence block-G sequence block, or G sequence block-P sequence block. The sequence structure is particularly preferably G sequence block-P sequence block-G sequence block, or G sequence block-P sequence block-P sequence block-G sequence block. In these structures, “-” indicates a direct link (between blocks) or another sequence (e.g., a linker sequence).

The polypeptide of the present invention may be terminated with a cell adhesive sequence. The cell adhesive sequence is not particularly limited, and various sequences can be used. The cell adhesive sequence may be one that does not have cell selectivity, such as RGDS (SEQ ID NO: 8), or one that has cell selectivity (e.g., REDV (SEQ ID NO: 20)). Examples of sequences that have cell selectivity include HHH, VVV, TTT, TGA, NNN, KKK, AAA, RRR, YYY, TTT, GAT, GGG, PGH, GQA, QGD, GIG, EKG, KGK, QGF, GMK, GLS, CAG, CNG, KGT, PLG, NRG, CSG, LGL, AVG, GHP, GLI, GVG, GPS, SPG, GPP, GIS, GYL, GEK, QGE, CNY, FPG, GAP, APG, GEC, LPG, GPR, PCG, GDV, IGG, CDG, AVA, FLM, GFD, GTP, GPY, VSG, DGR, GIT, GFL, ASG, GCP, NQG, SGL, GGA, PDG, QAL, GLK, GSP, GEP, GNS, AKG, DGY, TOP, VGP, SLW, AAG, AGA, ARG, GRD, EGF, GSC, PGQ, HSQ, EAP, RGP, PGD, CNI, GFG, GPT, GDO, KGE, PFI, QOGP, SYW, LPGFPGLK (SEQ ID NO: 9), LPGFPGTP (SEQ ID NO: 10), GPPGLSGPP (SEQ ID NO: 11), FPGPPGPP (SEQ ID NO: 12), LPGLPGPP (SEQ ID NO: 13), FPGLPGPP (SEQ ID NO: 14), GPPGPPGSPG (SEQ ID NO: 15), LPGPPGPP (SEQ ID NO: 16), FPGSPGFPG (SEQ ID NO: 17), GSPGLPGTP (SEQ ID NO: 18), and IGLSGEKG (SEQ ID NO: 19), and the like.

When the polypeptide of the present invention contains a cell adhesive sequence, the cell adhesive sequence is positioned at a terminus or the termini (the N-terminus and/or the C-terminus) of the polypeptide of the present invention, preferably at the C-terminus. A cell adhesive sequence may be positioned at either the N-terminus or the C-terminus, or both. In a preferred embodiment of the present invention, a cell adhesive sequence is positioned at only one terminus.

When the polypeptide of the present invention contains a cell adhesive sequence, the elastin-like block peptide sequence and the cell adhesive sequence may be directly linked, or another amino acid sequence (e.g. a sequence that is 3 to 30, 7 to 25, or 10 to 20 amino acids in length) may be interposed between the two. The other amino acid sequence is preferably a hydrophilic amino acid-rich sequence from the view point of cell adhesion. In other words, it is preferred that the hydrophilic amino acid-rich sequence is positioned on the side opposite to the end of the cell adhesive sequence. The hydrophilic amino acid-rich sequence is not particularly limited as long as it is a sequence in which the proportion of hydrophilic amino acids is high. Examples include amino acid sequences that are, for example, 3 to 15 or 5 to 10 amino acids in length and contain hydrophilic amino acids in an amount of, for example, 50% or more, 60% or more, 70% or more, 80% or more, or 90% or more. The hydrophilic amino acid-rich sequence is preferably a histidine consecutive sequence (His tag sequence) from the viewpoint of facilitating the purification of the polypeptide of the present invention. The other amino acid sequence preferably contains an amino acid having a functional group that can be used in a crosslinking reaction (e.g., lysine or cysteine).

The polypeptide of the present invention may be chemically modified as long as the thixotropic gel forming properties are not significantly impaired. The presence or absence of thixotropic gel forming properties can be evaluated according to Test Examples 2 and 3 described later.

The polypeptide of the present invention may have a carboxyl group (—COOH), carboxylate (—COO⁻), amide (—CONH₂), or ester (—COOR) at the C-terminus.

“R” in the ester is, for example, a C₁₋₆ alkyl group such as methyl, ethyl, n-propyl, isopropyl, or n-butyl; a C₃₋₈ cycloalkyl group such as cyclopentyl or cyclohexyl; a C₆₋₁₂ aryl group such as phenyl or α-naphthyl; a phenyl-C₁₋₂ alkyl group such as benzyl or phenethyl; a C₇₋₁₄ aralkyl group such as an α-naphthyl-C₁₋₂ alkyl group such as α-naphthyl methyl; or a pivaloyloxymethyl group.

The polypeptide of the present invention may have an amidated or esterified carboxyl group (or carboxylate), which is not the carboxyl group at the C-terminus. The ester in this case may be, for example, the esters of the C-terminus described above.

The polypeptide of the present invention further encompasses polypeptides having the amino group of the N-terminal amino acid residue protected by a protective group (e.g., a C₁₋₆ acyl group including a C₁₋₆ alkanoyl such as a formyl group and an acetyl group); polypeptides having the N-terminal glutamine residue pyroglutamated that can be formed due to cleavage in vivo; polypeptides having a substituent (e.g., —OH, —SH, an amino group, an imidazole group, an indole group, and a guanidino group) on a side chain of an amino acid in the molecule protected by an appropriate protective group (e.g., a C₁₋₆ acyl group including a C₁₋₆ alkanoyl group such as a formyl group and an acetyl group); conjugated proteins such as glycoproteins having a sugar chain bonded thereto; and the like.

The polypeptide of the present invention may be in the form of a pharmaceutically acceptable salt formed with an acid or base. The salt can be any pharmaceutically acceptable salt, and can be either an acid salt or a basic salt. Examples of acid salts include inorganic acid salts, such as hydrochloride, hydrobromide, sulfate, nitrate, and phosphate; organic acid salts, such as acetate, propionate, tartrate, fumarate, maleate, malate, citrate, methanesulfonate, and para-toluenesulfonate; amino acid salts, such as aspartate and glutamate; and the like. Examples of basic salts include alkali metal salts, such as sodium salts and potassium salts; alkaline-earth metal salts, such as calcium salts and magnesium salts; and the like.

The polypeptide of the present invention may be in the form of a solvate. The solvent can be any pharmaceutically acceptable solvent, and may be, for example, water, ethanol, glycerol, or acetic acid.

The polypeptide of the present invention can be easily produced according to a known genetic engineering method. For example, the polypeptide of the present invention can be produced using PCR, restriction enzyme cleavage, a DNA ligation technique, an in vitro transcription/translation technique, a recombinant protein production technique, etc.

The composition of the present invention comprises the polypeptide of the present invention and is in gel form or gel-forming sol form.

A gel composition that comprises the polypeptide of the present invention can be obtained by dissolving the polypeptide of the present invention in a solvent at a temperature lower than the LCST (e.g., 0 to 15° C., 2 to 10° C.), heating the resulting solution to the LCST or higher (e.g., 30 to 50° C., 30 to 45° C., 30 to 40° C.), and allowing the solution to stand for a certain period of time (e.g., 1 to 300 hours, preferably 8 to 120 hours, more preferably 12 to 80 hours). As the solvent, water is used. A mixed solvent of water and an organic solvent can also be used.

From the viewpoint of gel-forming properties, the concentration of the polypeptide of the present invention in the composition of the present invention is, for example, 0.2 w/v % or more, preferably 0.3 w/v % or more, and more preferably 0.35 w/v % or more. From the viewpoint of easily allowing other components (e.g., components necessary or suitable for cell culture) to permeate the gel composition of the present invention, the concentration is, for example, 3.0 w/v % or less, preferably 2.0 w/v % or less, more preferably 1.5 w/v % or less, even more preferably 1.0 w/v % or less, and still even more preferably 0.8 w/v % or less.

The composition in gel-forming sol form that comprises the polypeptide of the present invention is, in other words, a sol composition having gel-forming ability. Since the gel composition of the present invention exhibits thixotropic properties, a sol composition (sol composition 1) can be obtained by applying mechanical strain (e.g. pipetting or vortexing) to the gel composition of the present invention. The sol composition 1 is gelated when the mechanical strain is no longer applied or becomes weak, and thus can be said to have gel-forming ability. Moreover, a sol composition (sol composition 2) can be obtained by dissolving the polypeptide of the present invention at a low concentration at which a gel is not formed, in a solvent. The sol composition 2 is gelated when the concentration of the polypeptide of the present invention increases due to evaporation of the solvent or the like, and thus can be said to have gel-forming ability. Furthermore, a sol composition (sol composition 3) can be obtained by dissolving the polypeptide of the present invention in a solvent at a low temperature at which a gel is not formed (a temperature lower than the LCST). The sol composition 3 is gelated when heated to a gel-forming temperature (the LCST or higher) and allowed to stand for a certain period of time, and thus can be said to have gel-forming ability.

The composition of the present invention preferably retains cells. By retaining cells, the elastic modulus of the composition in gel form can be significantly improved. The manner of retention is not particularly limited, and it is preferred that cells are suspended in the composition of the present invention.

The organism species from which the cells are derived is not particularly limited, and examples include bacteria such as Enterobacteriaceae bacteria, fungi such as yeast, animals, and plants. Examples of animals include various mammals such as humans, monkeys, mice, rats, dogs, cats, rabbits, pigs, horses, bovine, sheep, goats, and deer, as well as non-mammalian vertebrates and invertebrates. In a preferred embodiment of the present invention, examples of organism species from which the target cells are derived include mammals, Escherichia coli, yeast, Bacillus subtilis, zebrafish, medaka, insects, and the like. The type of cell is also not particularly limited, and examples include cells derived from various tissues or having various properties, such as blood cells, hematopoietic stem cells/progenitor cells, gametes (sperm, egg), fibroblasts, epithelial cells, vascular endothelial cells, nerve cells, hepatic cells, keratinocytes, muscle cells, epidermal cells, endocrine cells, ES cells, iPS cells, tissue stem cells, and cancer cells.

When the composition of the present invention retains cells, the cell concentration is preferably 1.0×10⁵ cells/mL or more, more preferably 2.0×10⁵ cells/mL or more, even more preferably 5.0×10⁵ cells/mL or more, and still even more preferably 1.0×10⁶ cells/mL or more, from the viewpoint of further improving the elastic modulus of the composition in gel form. The upper limit of the cell concentration may be, for example, 1.0×10⁸ cells/mL, 1.0×10⁷ cells/mL, or 5.0×10⁶ cells/mL.

The method for retaining cells in the composition of the present invention is not particularly limited. Cells can be easily retained in the composition of the present invention by suspending the cells in the composition of the present invention in sol form. The resulting sol composition can be gelated to enable three-dimensional culture of cells.

The composition of the present invention may comprise one or more other components in addition to the above. The other components include, but are not particularly limited to, components necessary or suitable for culturing cells. Examples of the other components include standard inorganic salts such as magnesium, calcium, potassium, zinc, and iron, buffers, sugars, vitamins, essential amino acids, nonessential amino acids, and the like. Specific examples include Dulbecco's modified Eagle's medium (DMEM), minimal essential medium (MEM), basal medium eagle (BME), RPMI1640, F-10, F-12, α-minimal essential medium (αMEM), Glasgow's minimal essential medium (GMEM), Iscove's modified Dulbecco's medium (IMDM), and the like. In addition to the above, the other components include serum, serum substitutes, extracellular matrices, other protein components, polysaccharides, and the like.

The content of the other components may be, for example, 50 parts by weight or less, preferably 30 parts by weight or less, more preferably 20 parts by weight or less, even more preferably 10 parts by weight or less, and still even more preferably 5 parts by weight or less, per 100 parts by weight of the polypeptide of the present invention.

When the composition of the present invention is in gel form, its shape is not particularly limited as long as it is a shape suitable as a substrate for cell culture or growth. The shape may be, for example, a cube, a rectangular parallelepiped, a cone, a pyramid, a cylinder, a prism, or the like, or a composite shape consisting of two or more shapes.

The dried product of the composition of the present invention is any product in which the moisture content has been reduced from the composition of the present invention. The dried product of the composition of the present invention may have a moisture content of, for example, 50 wt % or less, 30 wt % or less, 20 wt % or less, 10 wt % or less, or 5 wt % or less. The dried product of the composition of the present invention can be used as a substrate for cell culture or growth by increasing the moisture content by adding water as necessary.

The substrate of the present invention may consist of the composition of the present invention or may be a composite of the composition of the present invention and one or more other substances. The other substances may be structures that can form part of, for example, a cell culture vessel. Examples of materials of the structures include glass, resins (e.g., polystyrene, polyethylene, polypropylene, poly(acrylic acid ester), poly(methacrylic acid ester), polyacrylamide, polyacrylonitrile, polyethylene terephthalate, poly(L-lactic acid), poly(glycolic acid), poly(ε-caprolactone), poly(ethylene glycol), and copolymers thereof), polypeptides (e.g., collagen, gelatin, casein, fibroin, keratin, laminin, integrin, fibronectin, and vitronectin), polysaccharides (e.g., cellulose, hyaluronic acid, chondroitin sulfate, starch, chitin, and chitosan), silica, silicon, metals (e.g., gold, silver, copper, iron, zinc, aluminum, nickel, and alloys or oxides thereof), and the like.

The substrate of the present invention is for use in cell culture or growth. Specifically, the substrate of the present invention is used for culturing or growing cells by retaining cells in the composition of the present invention or by placing cells on the surface of the composition of the present invention. From this viewpoint, in an embodiment of the present invention, the present invention relates to a cell culture or growth method comprising culturing or growing cells using the substrate of the present invention. The substrate of the present invention can also be suitably used as a bioink.

The term “culture or growth” as used herein includes not only cell division and growth, cell differentiation, etc., but also everything in which cells are maintained in a viable state. The term “cells” means not only cells that are individually separated, but also cells in cell populations in tissues, cell masses, spheroids, organoids, etc. From this viewpoint, in an embodiment of the present invention, the present invention relates to a cell population obtainable by the cell culture or growth method described above.

Components necessary or suitable for cell culture or growth can be supplied to cells by incorporating them beforehand in the composition of the present invention or by immersing the composition of the present invention that retains cells or has cells placed on its surface in a culture medium. Since the composition of the present invention can maintain a gel state even at low concentrations of the polypeptide of the present invention, nutritional components can be permeated into the composition of the present invention even by the latter method.

In a preferred embodiment, the substrate of the present invention can be used for three-dimensional culture or three-dimensional growth of cells. In three-dimensional culture or three-dimensional growth, for example, cells are dispersed in the composition of the present invention in gel form and cultured or grown. In this case, components necessary for culture or growth (culture medium components) can be supplied to cells by bringing a solution containing the culture medium components (culture medium) into contact with the composition of the present invention in gel form or by using the composition of the present invention in gel form containing the culture medium components. This allows, for example, the formation of spheroids. In spheroid formation, a spheroid can be obtained by culturing or growing cells for, for example, 3 days or more, preferably 3 to 10 days. After culture or growth, mechanical strain can be applied to the composition of the present invention in gel form to cause sol-gel transition, and the cells in the sol can be recovered.

In a preferred embodiment, the substrate of the present invention can be used for two-dimensional culture or two-dimensional growth of cells. In two-dimensional culture or two-dimensional growth, for example, cells are placed on the surface of the composition of the present invention in gel form and cultured or grown. This allows, for example, the formation of a cell sheet. After culture or growth, the cells or cell sheet can be detached by leaving the cells or cell sheet at a relatively low temperature (e.g., 25° C. or lower, preferably 22° C. or lower). Alternatively, after culture or growth, mechanical strain can be applied to the composition of the present invention in gel form to cause sol-gel transition, and the cells or cell sheet can be detached.

The substrate of the present invention can also be suitably used as a bioink for cellular 3D bioprinting. For example, when cells are suspended in the composition of the present invention in sol form and spatially arranged, the desired three-dimensional tissue structure can be obtained by sol-gel transition.

The substrate of the present invention can be used not only as a cell culture substrate, but also as a cell growth substrate for forming artificial blood vessels, a cell growth substrate in wound treatment, and the like.

### EXAMPLES

The present invention is described below in detail with reference to Examples; however, the present invention is not limited to these Examples.

### Test Example 1: Synthesis of Polypeptides

The following two polypeptides (GPG1 and GPG3) were synthesized: (GPG1) a polypeptide (SEQ ID NO: 5) comprising an elastin-like block peptide sequence that comprises a G sequence block consisting of (SEQ ID NO: 3: VGGVG)₅, a P sequence block consisting of (SEQ ID NO: 4: VPGXG)₂₅, wherein each X is the same or different and represents V or F, a G sequence block consisting of (SEQ ID NO: 3: VGGVG)₅ in this order from the N-terminal side, each block being linked via a linker sequence; and (GPG3) a polypeptide (SEQ ID NO: 6) in which a cell adhesive sequence is positioned on the C-terminal side of GPG1. GPG1 and GPG3 are polypeptides consisting of previously reported amino acid sequences.

Specifically, E. coli BLR (DE3) strain was transformed with plasmid DNAs encoding these polypeptides to express the polypeptides. The polypeptides were obtained by purification using metal ion affinity chromatography using a His-tag. SDS-PAGE and MALDI-TOF-MS confirmed that the polypeptides of interest were obtained.

Each of the recovered peptide solutions was individually placed in Spectra/Por (registered trademark) 1 Dialysis Membrane MWCO: 6-8,000 (Spectrum Laboratories), followed by dialysis in 2 L of ultrapure water at 4° C. Ultrapure water was exchanged twice every hour (minimum time), then twice every 2 hours, and then once after 1.5 hours, a total of five times, for the dialysis. After the dialysis, each of the peptide solutions was individually filtered through Minisart (registered trademark) Syringe Filter 0.2 μm (Sartorius Stedim Biotech) attached to syringes to remove insolubilized peptides, followed by freezing at −80° C. overnight. Thereafter, freeze-drying was performed with an FDU-1200 freeze dryer (Tokyo Rikakikai) to obtain powders of two peptides, i.e., GPG1 and GPG3.

### Test Example 2: Gelation of Polypeptide Solutions

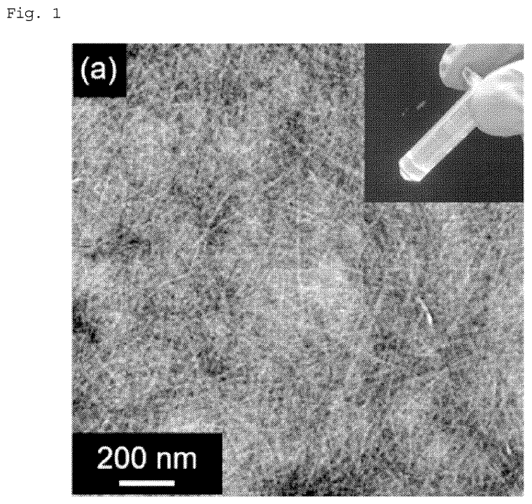

GPG1 was dissolved in a 10 w/v % aqueous sucrose solution to a final concentration of 0.5 wt %. The resulting GPG1 solution was heated and maintained at 37° C. The GPG1 solution was then allowed to stand. After 1 day, the GPG1 solution had become a transparent gel. TEM observation of the resulting gel confirmed the formation of nanofibers with a diameter of 10 nm or less (FIG. 1).

The same test was also performed for GPG3, and the obtained results were similar to those of GPG1.

### Test Example 3: Thixotropic Properties of Gels

The GPG1 gel obtained in Test Example 2 was subjected to rheological measurement at a constant frequency of 1 Hz while alternating between 100% shear strain conditions and 0.5% shear strain conditions. The rheological measurement was performed using a Modular Compact Rheometer MCR302 (Anton Paar) and 1° cone plate (diameter: 25 mm) with a gap of 0.048 mm. During all measurements, Prowipe impregnated with ultrapure water was placed around the measurement stand and covered with a solvent trap cover to prevent drying of the samples. Furthermore, the temperature was maintained at 37° C. using a Peltier system contained in the rheometer. Each prepared sample was placed in an appropriate amount on the measurement stand with a spatula, and the jig was lowered to the target gap. The storage modulus (G′) and loss modulus (G″) were measured.

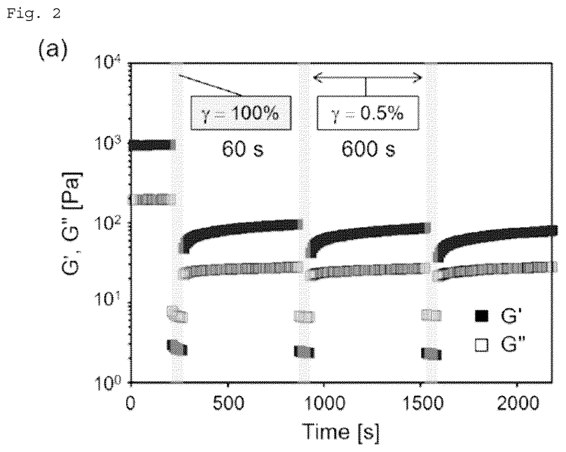

FIG. 2 show the results. The GPG1 gel was found to be capable of repeated reversible breakdown of the gel under mechanical strain and recovery under static conditions, i.e., the GPG1 gel was found to exhibit thixotropic properties (self-healing properties).

The same test was also performed for the GPG3 gel, and the obtained results were similar to those of the GPG1 gel.

### Test Example 4: Rheology at the Time of Initial Gelation

GPG1 was dissolved in a 10 w/v % aqueous sucrose solution to a final concentration of 0.4 w/v %, and the resulting GPG1 solution was used as a sample.

The rheological measurement method was the same as in Test Example 3. The sample in a solution state at 4° C. was set in an MCR302 rheometer adjusted to 37° C. for dynamic viscoelasticity measurement. The dynamic viscoelasticity was measured in three items: time dispersion, frequency dispersion, and strain dispersion, and the storage modulus (G′) and loss modulus (G″) were measured for each. First, time-dispersion measurement was performed on one sample at a constant strain of 1% and a constant frequency of 1 Hz, with one point measured every 30 seconds, i.e., 120 points for a total of 60 minutes. Subsequent frequency dispersion measurement was performed at a constant strain of 1% and in the angular frequency range of 100 to 0.1 rad/s. Finally, strain dispersion measurement was performed at a constant frequency of 1 Hz and in the shear strain range of 0.1 to 100%. In all of the samples, it was observed from the results of the strain dispersion measurement that 1% strain was in the linear region.

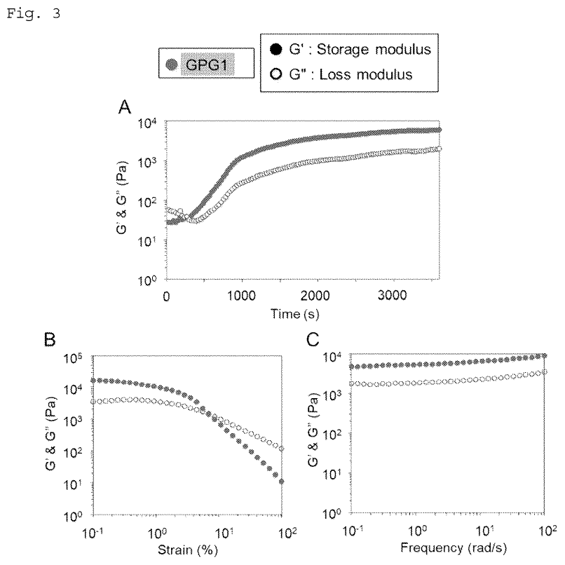

FIG. 3 shows the results. In the time dispersion measurement shown in FIG. 3A, at the first point immediately after the start of the measurement (after 30 seconds), G′ was 28 Pa, and G″ was 56 Pa, i.e., G″ exceeded G′. Over time, G′ gradually increased, and G″ gradually decreased. G′ exceeded G″ at 300 seconds. G′ continued to increase, and after about 1000 seconds, the rate of increase became slower from around 1000 Pa. G′ reached about 6000 Pa at the end of the measurement. G″ showed behavior similar to that of G′, finally reaching about 2000 Pa. In the strain dispersion measurement shown in FIG. 3B, G′ gradually decreased from 0.1% strain to about 1.46% strain and rapidly decreased after 1.46% strain. On the other hand, G″ showed an increasing trend from 0.1% strain to about 0.5% strain and decreased after 0.5% strain; however, its slope was gentler than that of G′. From 0.1% strain to about 7% strain, elastic behavior, in which G′ exceeded G″, was exhibited, and after about 7% strain, viscous behavior, in which G″ exceeded G′, was exhibited. In the frequency dispersion measurement shown in FIG. 3C, elastic behavior, in which G′ exceeded G″, was exhibited over the entire measured frequency range of 0.1 to 100 rad/s. Both G′ and G″ showed changes on a scale of 10″ Pa, and each value gradually increased from the low frequency region to the high frequency region. Furthermore, the loss tangent tan δ, defined as tan δ (=G″/G′), was 0.330 to 0.390.

The same test was also performed for the GPG3 gel, and the obtained results were similar to those of the GPG1 gel.

### Test Example 5: Rheology at the Time of Re-Gelation (without Cells)

GPG1 was dissolved in a 10 w/v % aqueous sucrose solution to a final concentration of 0.8 w/v %, and the resulting GPG1 solution was allowed to stand at 45° C. for one day for initial gelation. The gel was solated by shaking with a vortex mixer (Scientific Industries) for 5 seconds. An equal amount of a 10 w/v % aqueous sucrose solution was added thereto to give a sol with a peptide concentration of 0.4 w/v %. Gelation of the sol by raising the temperature again is hereinafter referred to as “re-gelation”. The rheological measurement method was the same as in Test Example 3. The GPG1 sol (peptide concentration: 0.4 w/v %) was set in an MCR302 rheometer adjusted to 37° C. immediately after removal from a 45° C. incubator, and dynamic viscoelasticity measurement was performed in the same manner as in Test Example 4.

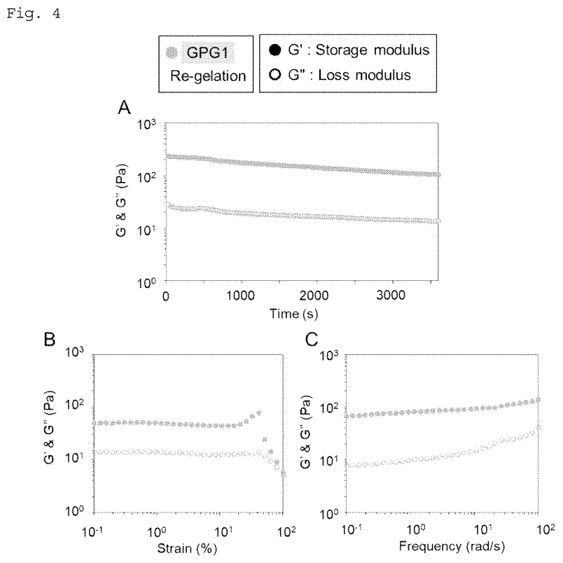

FIG. 4 shows the results. In the time dispersion measurement shown in FIG. 4A, G′ always exceeded G″ from immediately after the start of the measurement to the end of the measurement. At the first point (after 30 seconds), G′ was 233 Pa, and G″ was 28 Pa. G′ and G″ continued to decrease with similar behavior throughout the measurement and at the end of the measurement, G′ was 105 Pa, and G″ was 13 Pa. In the strain dispersion measurement shown in FIG. 4B, the values of G′ and G″ were in the linear range, in which they were almost constant, up to 17% strain. When further strain was applied, G′ increased and then rapidly decreased when more than 41% strain was applied. G″ continued to be in the linear range up to 41% strain and then gradually decreased. No reversal of the dominance relationship between G′ and G″ was observed. In the frequency dispersion measurement shown in FIG. 4C, elastic behavior, in which G′ exceeded G″, was exhibited over the entire measured frequency range of 0.1 to 100 rad/s. Gradual increases in G′ and G″ were observed from the low frequency region to the high frequency region. G′ showed changes on a scale of 101 to 10², G″ showed changes on a scale of 10⁰ to 10¹, and tan δ (=G″/G′) was 0.116 to 0.295.

The same test was also performed for the GPG3 gel, and the obtained results were similar to those of the GPG1 gel.

### Test Example 6: Rheology at the Time of Re-Gelation (with Cells)

GPG1 was dissolved in a 10 w/v % aqueous sucrose solution to a final concentration of 0.8 w/v %, and the resulting GPG1 solution was allowed to stand at 45° C. for one day for initial gelation. The gel was solated by shaking with a vortex mixer (Scientific Industries) for 5 seconds. An equal amount of a cell suspension prepared by suspending Panc-1 cells in a 10 w/v % aqueous sucrose solution was added thereto to give a sol with a peptide concentration of 0.4 w/v % and a cell concentration of 2.0×10⁶ cells/mL. Measurement was performed in the same manner as in Test Example 5, using the sol.

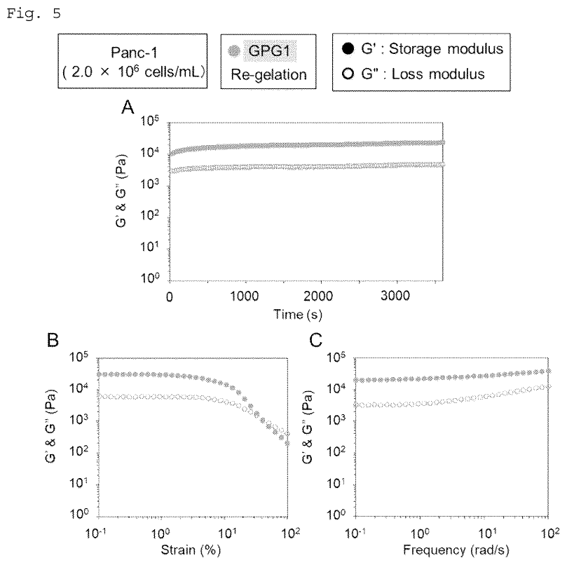

FIG. 5 shows the results. In the time dispersion measurement shown in FIG. 5A, G′ always exceeded G″ from immediately after the start of the measurement to the end of the measurement. At the first point (after 30 seconds), G′ was 10578 Pa, and G″ was 2913 Pa. From immediately after the start of the measurement, G′ was in equilibrium and continued to gradually increased. G″ showed similar behavior. At the end of the measurement, G′ was 23487 Pa, and G″ was 4742 Pa. In the strain dispersion measurement shown in FIG. 5B, the values of G′ and G″ were in the linear range, in which they were almost constant, up to about 2.84% strain. When further strain was applied, G′ rapidly decreased, and G″ gradually decreased. As a result, the dominance relationship between G′ and G″ was reversed at around 41% strain. In the frequency dispersion measurement shown in FIG. 5C, elastic behavior, in which G′ exceeded G″, was exhibited over the entire measured frequency range of 0.1 to 100 rad/s. Gradual increases in G′ and G″ were observed from the low frequency region to the high frequency region. G′ showed changes on a scale of 104, G″ showed changes on a scale of 103, and tan δ (=G″/G′) was 0.155 to 0.326.

These results and the results of Test Example 5 without cells together showed that by suspending cells in the GPG sol/gel, the elastic modulus of the gel was significantly improved.

The same test was also performed for the GPG3 gel, and the obtained results were similar to those of the GPG1 gel. No improvement in the gel elastic modulus due to suspending cells was observed in the case of a collagen gel.

### Test Example 7: Three-Dimensional Cell Culture Using Thixotropic Properties of Gel

50 μL of a cell suspension sol obtained in the same manner as in Test Example 6 was added dropwise to each well of a 96-well plate (Corning, Inc., U.S.A.) and allowed to stand in an incubator at 37° C. for 10 minutes to cause gelation, thereby preparing a cell layer. 100 μL of cell culture medium (DMEM) was added dropwise from above, and culture was performed in an incubator at a CO₂ concentration of 5% and 37° C. for each period. The time when the cell layer was prepared was set as day 0, and on days 1, 3, and 5, 50 μL of the supernatant cell culture medium was removed, and 50 μL of fresh cell culture medium was added dropwise to exchange the cell culture medium. On days 0, 2, 4, and 7, the gel was solated by pipetting with a micropipette and dispersed in PBS.

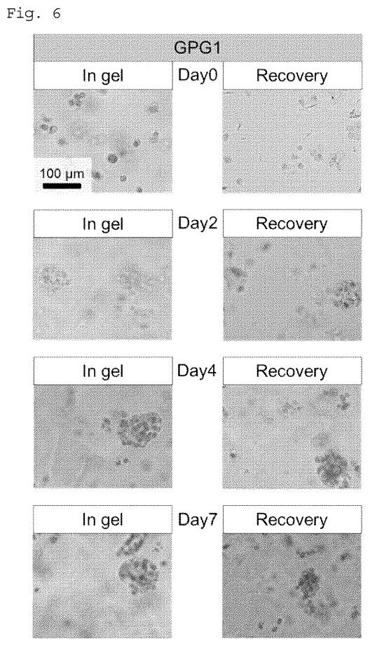

The cells in the gel and the cells dispersed in PBS after solation on days 0, 2, 4, and 7 were observed with a phase-contrast microscope. FIG. 6 shows the images obtained by observation. It was found that spheroids were formed by culturing using the GPG gel. It was also found that cells were easily recovered without destroying spheroids by using the thixotropic properties of the GPG gel.

Spheroids were also formed by three-dimensional culture using a collagen gel, but could not be recovered by pipetting and could be recovered after enzyme treatment with collagenase.

### Test Example 8: Two-Dimensional Cell Culture

NIH/3T3 cells were cultured on the GPG1 gel at 37° C. by two-dimensional culture. After culture, the cells were left at room temperature (about 15° C.) for about 10 minutes and then detached in a sheet-like form. Since GPG is temperature responsive, GPO becomes hydrophilic at room temperature, which is thought to be why the cells were detached. These results showed that a cell sheet can be easily recovered by culturing cells on the GPG gel by two-dimensional culture.

### Test Example 9: Three-Dimensional Cell Culture Using Thixotropic Properties of Gel (Myoblast C2C12)

GPG3 was dissolved in 10 w/v % sucrose to a final concentration of 0.8 w/v %, and the resulting GPG3 solution was allowed to stand at 45° C. for 2 days for initial gelation. The gel was solated by shaking with a vortex mixer for 5 seconds. 50 μL of the sol was added dropwise to a cell culture vessel using a micropipette, followed by mixing therewith 50 μL of a suspension of mouse-derived myoblast cell line C2C12 (Riken CELL BANK) (4×10⁶ cells/mL) dispersed in 10 w/v % sucrose, for re-gelation, thereby preparing a GPG3 gel with a final GPG3 concentration of 0.4 w/v % and a cell concentration of 2×10⁶ cells/mL. The GPG3 gel was overlayed with 1 mL of DMEM medium containing 10 v/v % fetal bovine serum (FBS), and culture was started at 37° C. and a 5% CO₂ concentration. Medium exchange was performed at 300 μL every day, and on day 3, the medium was switched to differentiation induction medium (DMEM medium containing 2 v/v % horse serum). After differentiation induction, culture was performed for 7 days.

The number of cells was evaluated with Cell Counting Kit-8 (CCK-8, DOJINDO) 4 hours and 3 days after the start of culture. The procedure was as follows. First, the medium was aspirated with a micropipette, and the remaining gel was solated by pipetting, and the sol was collected in a microtube. The cells were precipitated at 1000 rpm using a tabletop centrifuge, 100 μL of the supernatant was collected in a 96-well plate as background, and the remaining supernatant was removed. The precipitated cells were dispersed in 150 μL of DMEM medium, and 100 μL of the dispersion was transferred to the 96-well plate. 10 μL of a CCK solution was added to each of the wells containing the background and the sample, and the plate was allowed to stand in a 37° C. incubator for 3 hours. Thereafter, the absorbance at 450 nm was measured with a microplate reader (EPOCH2; BioTek). The cell growth rate was estimated by assuming that the difference in absorbance between the sample and background was proportional to the number of viable cells (Table 1).

TABLE 1

| Relative number of viable cells | | |
|---|---|---|
| After 4 hours of culture | After 3 days of culture | Growth rate |
| 0.927 | 2.967 | 3.2 |

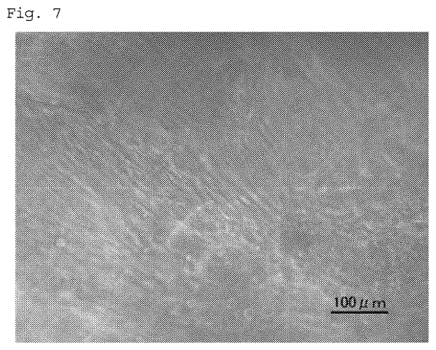

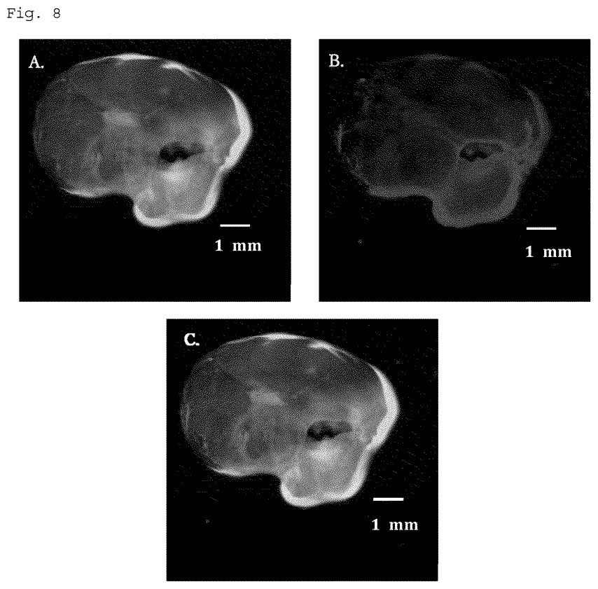

Seven days after the start of differentiation induction, muscle-like cells were observed in the gel with a phase-contrast microscope (BZX700, KEYENCE) (FIG. 7). The entire gel was immunostained with α-actinin, which is a skeletal muscle-specific expression marker (FIG. 8), suggesting that myoblasts can be cultured and differentiate into skeletal muscle cells in the GPG3 gel.

The immunostaining procedure is as follows. The medium was aspirated using an aspirator, and the gel was washed twice with 300 μL of phosphate-buffered saline (PBS). 400 μL of 4% paraformaldehyde phosphate buffer was added, and the mixture was allowed to stand at 4° C. for 30 minutes and then washed twice with 300 μL of PBS. Furthermore, polyoxyethylene (10) octylphenyl ether was diluted with PBS to prepare a 0.5% concentration solution, which was used as a Triton X-100 solution. 400 μL of this solution was added onto the gel, and the gel was allowed to stand at 4° C. for 25 minutes and then washed twice with 300 μL of PBS. Bovine serum-derived Cohn fraction V was diluted with PBS to prepare a 1.0% concentration solution, which was used as a BSA solution. 400 μL of the BSA solution was added onto the gel, and the gel was allowed to stand at room temperature for 1 hour and washed twice with 300 μL of PBS. 400 μL of Sarcomeric alpha Actinin Monoclonal Antibody (EA-53, Thermo Fisher Scientific) diluted 200-fold with the BSA solution was added thereto as a primary antibody, followed by allowing it to stand at 4° C. for 16 hours and washing twice with 300 μL of PBS. Furthermore, Goat anti-Mouse IgG (H+L) Highly Cross-Adsorbed Secondary Antibody, Alexa Fluor™ Plus 488 (Thermo Fisher Scientific) was added to the BSA solution so that the concentration was 0.1%, thereby preparing a secondary antibody. 400 μL of the secondary antibody was added onto the gel, and the gel was allowed to stand at room temperature under light-shielding conditions for 1 hour and washed twice with 300 μL of PBS. In addition, 4 drops of NucBlue™ Fixed Cell ReadyProbes™ reagent (Thermo Fisher Scientific) were added to 2 mL of PBS to prepare a nuclear staining solution. 400 μL of the nuclear staining solution was added onto the gel, and the gel was allowed to stand at room temperature under light-shielding conditions for 5 minutes. The gel was washed once with 300 μL of PBS and observed with a fluorescence microscope (BZ-X700, KEYENCE).

1. A cell culture or growth substrate comprising a composition in gel form or gel-forming sol form, or a dried product of the composition, the composition comprising a polypeptide comprising an elastin-like block peptide sequence that comprises a G sequence block consisting of (SEQ ID NO: 1: X¹GGX²G)ₙ, wherein each X¹ is the same or different and represents V or L, each X² is the same or different and represents V or L, and n represents an integer of 4 or more, and a P sequence block consisting of (SEQ ID NO: 2: VPGX³G)ₘ, wherein each X³ is the same or different and represents any amino acid, and m represents an integer of 5 or more.

2. The cell culture or growth substrate according to claim 1, wherein the sequence structure of the elastin-like block peptide sequence is, from the N-terminal side, G sequence block-P sequence block-G sequence block, G sequence block-P sequence block-P sequence block-G sequence block, P sequence block-G sequence block, or G sequence block-P sequence block.

3. The cell culture or growth substrate according to claim 1, for use in three-dimensional culture or three-dimensional growth of cells.

4. The cell culture or growth substrate according to claim 1, for use in two-dimensional culture or two-dimensional growth of cells.

5. The cell culture or growth substrate according to claim 1, wherein the composition retains cells.

6. The cell culture or growth substrate according to claim 1, wherein the concentration of the polypeptide in the composition is 0.2 w/v % or more.

7. The cell culture or growth substrate according to claim 1, wherein the concentration of the polypeptide in the composition is 0.2 to 3 w/v %.

8. The cell culture or growth substrate according to claim 1, for use by solating the composition in gel form and/or for use by gelating the composition in sol form.

9. A cell culture or growth method comprising culturing or growing cells using the cell culture or growth substrate according to claim 1.

10. A cell population obtainable by the cell culture or growth method according to claim 9.
